(19) United States
(12) Patent Application Publication
(10) Pub. No.: US 2008/0039494 A1
(43) Pub. Date: Feb. 14, 2008

(54) 8-Phenyl-5,6,7,8-Hydroquinoline Tachykinin Receptor Antagonists

(76) Inventors: Jianming Bao, Scotch Plains, NJ (US); Robert J. DeVita, Westfield, NJ (US); Jinlong Jiang, Scotch Plains, NJ (US); Sander G. Mills, Scotch Plains, NJ (US)

Correspondence Address: MERCK AND CO., INC, P O BOX 2000, RAHWAY, NJ, 07065-0907, US

(21) Appl. No.: 11/667,908

(22) PCT Filed: Nov. 29, 2005
(86) PCT No.: PCT/US05/43002

§ 371 Date: May 16, 2007

Related U.S. Application Data
(60) Provisional application No. 60/632,789, filed on Dec. 3, 2004.

(51) Int. Cl. A61K 31/47; A61P 29/00; C07D 217/22

(52) U.S. Cl. 514/309; 546/141

(57) ABSTRACT

The present invention is directed to certain 8-phenyl-5,6,7,8-hydroquinolinecompounds which are useful as neurokinin-1 (NK-1) receptor antagonists, and inhibitors of tachykinin and in particular substance P. The invention is also concerned with pharmaceutical formulations comprising these compounds as active ingredients and the use of the compounds and their formulations in the treatment of certain disorders, including emesis, urinary incontinence, depression, and anxiety.

### BACKGROUND OF THE INVENTION

Substance P is a naturally occurring undecapeptide belonging to the tachykinin family of peptides, the latter being so-named because of their prompt contractile action on extravascular smooth muscle tissue. The tachykinins are distinguished by a conserved carboxyl-terminal sequence. In addition to substance P, the known mammalian tachykinins include neurokinin A and neurokinin B. The current nomenclature designates the receptors for substance P, neurokinin A, and neurokinin B as neurokinin-1 (NK-1), neurokinin-2 (NK-2), and neurokinin-3 (NK-3), respectively. Tachykinin, and in particular substance P, antagonists are useful in the treatment of of clinical conditions which are characterized by the presence of an excess of tachykinin, in particular substance P, activity, including disorders of the central nervous system, nociception and pain, gastrointestinal disorders, disorders of bladder function and respiratory diseases. Attempts have been made to provide antagonists for the receptors of substance P and other tachykinin peptides in order to more effectively treat the various disorders and diseases mentioned above.

### SUMMARY OF THE INVENTION

The present invention is directed to certain quinoline compounds which are useful as neurokinin-1 (NK-1) receptor antagonists, and inhibitors of tachykinin and in particular substance P. The invention is also concerned with pharmaceutical formulations comprising these compounds as active ingredients and the use of the compounds and their formulations in the treatment of certain disorders, including emesis, urinary incontinence, depression, and anxiety.

### DETAILED DESCRIPTION OF THE INVENTION

The present invention is directed to compounds of the formula I: 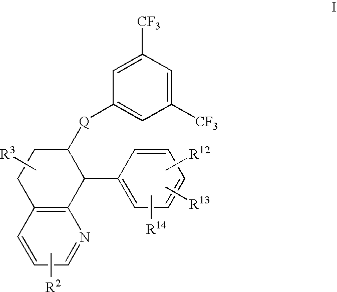
and N-oxides thereof, wherein:
Q is selected from the group consisting of:
(1) —O—CH₂—,
(2) —O—CH(CH₃)—,
(3) —O—CH(CH₂OH)—,
(4) —O—(CO)—, and
(5) —O—(C═CH₂)—;
   R² and R³ are independently selected from the group consisting of:
(1) hydrogen,
(2) C₁₋₆ alkyl, which is unsubstituted or substituted with one or more of the substituents selected from:
   (a) hydroxy,
   (b) oxo,
   (c) C₁₋₆ alkoxy,
   (d) phenyl-C₁₋₃ alkoxy,
   (e) phenyl,
   (f) halo,
   (g) —NR⁹R¹⁰, wherein R⁹ and R¹⁰ are independently selected from:
      (I) hydrogen,
      (II) C₁₋₆ alkyl,
      (III) phenyl,
      (IV) (C₁₋₆ alkyl)-phenyl,
      (V) (C₁₋₆ alkyl)-hydroxy, and
      (VI) (C₁₋₆ alkyl)-(C₁₋₄ alkoxy), or where —NR⁹R¹⁰ forms a morpholine, piperidine or quinuclidine ring
   (h) —NR⁹—COR¹¹, wherein R¹¹ is independently selected from:
      (I) hydrogen,
      (II) C₁₋₆ alkyl,
      (III) phenyl,
      (IV) (C₁₋₆ alkyl)-phenyl,
      (V) (C₁₋₆ alkyl)-hydroxy, and
      (VI) (C₁₋₆ alkyl)-(C₁₋₄ alkoxy),
   (j) —NR⁹—CO₂R¹¹,
   (k) —CO—NR⁹R¹⁰,
   (l) —COR¹¹,
   (m) —CO₂R¹¹,
(3) hydroxy,
(4) C₁₋₆alkoxy,
(5) oxo,
(6) halo,
(7) —CN,
(8) —CF₃,
(9) —NR⁹R¹⁰,
(10) —NR⁹—COR¹¹,
(11) —NR⁹—CO₂R¹¹,
(12) —CO—NR⁹-COR¹¹,
(13) —COR¹¹,
(14) —O—(CO)R¹¹,
(15) —CO₂R¹¹,
(16) -imidazolyl, and
(17) -triazolyl;
   R¹², R¹³ and R¹⁴ are independently selected from the group consisting of:
(1) hydrogen,
(2) halo, and
(3) C₁₋₆ alkyl;
   and pharmaceutically acceptable salts thereof and individual enantiomers and diastereomers thereof.

An embodiment of the present invention includes compounds of the formula Ia: 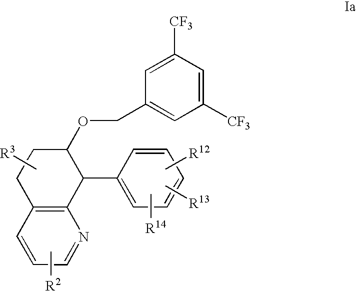
and N-oxides thereof, wherein R², R³, R¹², R¹³ and R¹⁴ are defined herein; and pharmaceutically acceptable salts thereof and individual enantiomers and diastereomers thereof.

Within this embodiment, the present invention includes compounds of the formula Ia′: 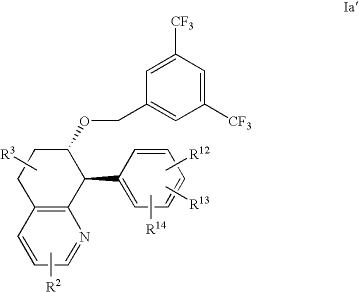
and N-oxides thereof, wherein R², R³, R¹², R¹³ and R¹⁴ are defined herein; and pharmaceutically acceptable salts thereof and individual enantiomers and diastereomers thereof.

An embodiment of the present invention includes compounds of the formula Ib: 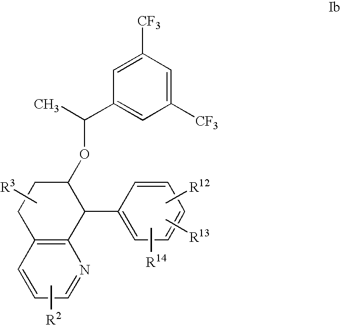
and N-oxides thereof, wherein R², R³, R¹², R¹³ and R¹⁴ are defined herein; and pharmaceutically acceptable salts thereof and individual enantiomers and diastereomers thereof.

Within this embodiment, the present invention includes compounds of the formula Ib′: 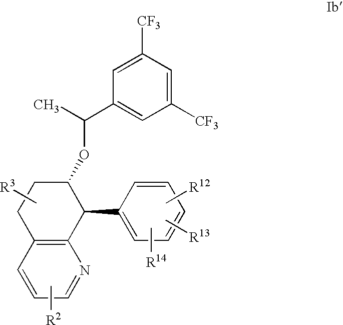
and N-oxides thereof, wherein R², R³, R¹², R¹³ and R¹⁴ are defined herein; and pharmaceutically acceptable salts thereof and individual enantiomers and diastereomers thereof.

An embodiment of the present invention includes compounds wherein R² is selected from the group consisting of:
(1) hydrogen,
(2) C₁₋₆ alkyl, which is unsubstituted or substituted with one or more of the substituents selected from:
   (a) morpholinyl,
   (b) —NH₂,
   (c) —NH(C₁₋₆ alkyl),
   (d) —N(C₁₋₆ alkyl)(₁₋₆ alkyl),
   (e) hydroxy,
   (f) —CO₂(C₁₋₆ alkyl),
(g) —NHCO(C₁₋₆ alkyl),
(h) —CO₂H, and
(i) triazolyl,
   (3) hydroxy,
   (4) halo,
   (5) —CO₂(C₁₋₆ alkyl),
   (6) —CO₂H, and
   (7) —CN.

Within this embodiment, the present invention includes compounds wherein R² is hydrogen.

Within this embodiment, the present invention includes compounds wherein R² is methyl.

An embodiment of the present invention includes compounds wherein R³ is hydrogen.

An embodiment of the present invention includes compounds wherein R³ is fluoro.

An embodiment of the present invention includes compounds wherein R¹² is fluoro, R¹³ is hydrogen or methyl, and R¹⁴ is hydrogen.

An embodiment of the present invention includes compounds wherein R¹² is 4-fluoro, R¹³ is hydrogen and R¹⁴ is hydrogen.

An embodiment of the present invention includes compounds wherein R¹² is 4-fluoro, R¹³ is 2-methyl and R¹⁴ is hydrogen.

An embodiment of the present invention includes compounds wherein the compound is present as an N-oxide on the pyridyl ring.

Specific embodiments of the present invention include a compound which is selected from the group consisting of the subject compounds of the Examples herein and pharmaceutically acceptable salts thereof and individual enantiomers and diastereomers thereof.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric fonns of these compounds. Formula I shows the structure of the class of compounds without preferred stereochemistry. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

There are several acceptable methods of naming the compounds discussed herein.

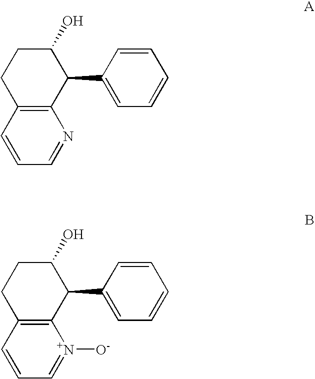

For example, the above compound A can be named as “(7S,8S)-8-phenyl-5,6,7,8-tetrahydroquinolin-7-ol.” The above compound B can be named as “(7S,8S)-8-phenyl-5,6,7,8-tetrahydroquinolin-7-ol 1-oxide” or alternatively “(7S,8S)-8-phenyl-5,6,7,8-tetrahydroquinolin-7-ol N-oxide.” The core structures A and B may be generally referred to as tetrahydroquinoline, or hydroquinoline and tetrahydroquinoline 1-oxide, tetrahydroquinoline N-oxide or hydroquinoline 1-oxide or hydroquinoline N-oxide compounds, respectively.

As appreciated by those of skill in the art, halo or halogen as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C i 6alkyl specifically includes methyl, ethyl, n-propyl, iso-propyl, n-butyl, iso-butyl, tert-butyl, pentyl and hexyl. A group which is designated as being independently substituted with substituents may be independently substituted with multiple numbers of such substituents.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethyl aamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are benzenesulfonic, citric, hydrobromic, hydrochloric, maleic, fumaric, succinic and tartaric acids. It will be understood that, as used herein, references to the compounds of the present invention are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

General reaction sequences to prepare the compounds of the present invention are outlined in the schemes below. The tetrahydroquinoline intermediates A may be reacted with benzaldehyde under thermal conditions to provide the 8-benzylidine intermediates B. The olefin of these compounds may be cleaved with ozone followed by reduction of the intermediate ozonide with reagents such as dimethyl sulfide to provide the 8-oxoquinolines C. The 8-oxo quinolines may be converted to the 8-vinyl triflate (trifluormethanesulfonate) by reaction with a strong base such as potassium bistrimethylsilylamide (K IMDS) followed by quenching with a triflating reagent such as trifluoromethanesulfonic anhydride. Resulting triflates D may be reacted with aryl metal reagents such as aryl boronic acids or aryl stannanes to provide the 8-aryl, 5,6-dihydroquinoline intermediates E. Alternatively, the heteroaryl ketones C may be reacted with aryl metal reagents such as aryllithium, cerium or magnesium reagents and the resulting crude aryl alcohols dehydrated in the presence of acid such as methanesulfonic acid to provide the intermediates of general structure E. The olefin of intermediates E may be hydroborated with reagents known to the skilled in the art and the intermediate organoboranes oxidized with hydrogen peroxide and base to provide the racemic trans-7-hydroxy-8-arylquinoline intermediates. These racemic intermediates may be further functionalized without separation of enantiomers by the reactions outlined below. The alcohols may be separated however, by chiral HPLC, to provide single enantiomers of the trans-7-hydroxy-8-arylquinoline intermediates F (only one enantiomer shown). The 7-hydroxyl group may be converted to ethers by a variety of reactions know to those skilled in the art such as “Williamson” ether synthesis, reaction with a trichloracetimidate under acid conditions or by the sequence of reactions shown in the scheme. The alcohol may acylated to form an ester. The resulting ester may be reacted with titanium olefination reagents such as the “Tebbe” reagent. The intermediate enol ether may then be hydrogenated to provide the 7-ether compounds of the present invention of the generalized structure I (wherein R¹ is the Q-((3,5-bis-trifluoro-methyl)phenyl) group and wherein R¹² includes R¹², R¹³ and R¹⁴).

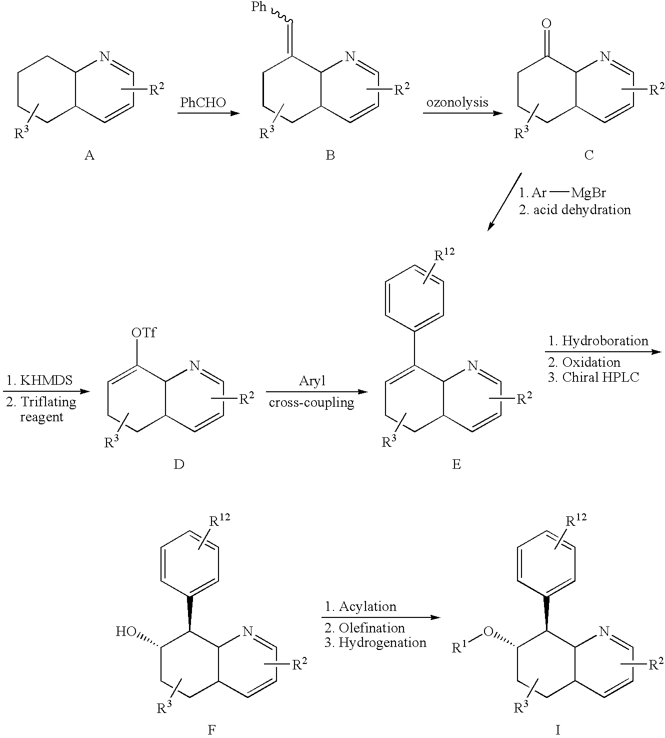

The cis 7-hydroxy-8-arylquinoline compounds may be prepared as outlined below. The chiral (or racemic) trans alcohols F may be reacted with an appropriate acid in the presences of an azodicarboxylate such diethylazodicarboxylate (DEAD) and a phosphine such as triphenylphosphine to provide the resulting ester G with inversion of stereochemistry. The intermediate ester G may be olefinated with the “Tebbe” reagent to provide the enol ether. Hydrogenation of the enolether will provide compounds of the general structure I of cis relative stereochemistry at the 7- and 8-positions.

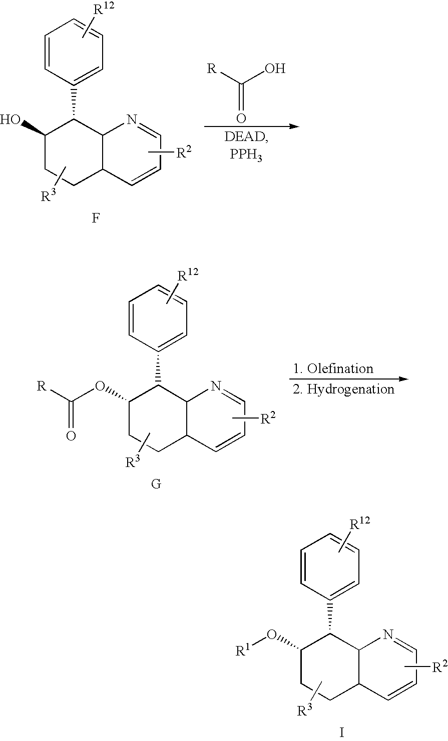

The compounds of the present invention of general structure I may be converted to N-oxide compounds of generalized structure II of the present invention by reaction with a variety of oxidizing reagents such as a per-acid such as m-CPBA.

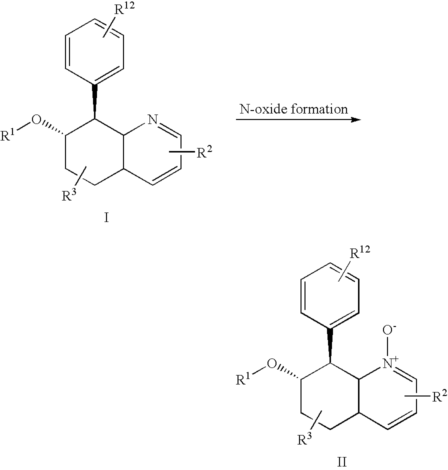

These generalized compounds I and II may serve as intermediates and may be further substituted or functionalized by reactions know to those skilled in the art and described in detail in the experimentals contained herein.

The compounds of the present invention are useful in the prevention and treatment of a wide variety of clinical conditions which are characterized by the presence of an excess of tachykinin, in particular substance P, activity. Thus, for example, an excess of tachykinin, and in particular substance P, activity is implicated in a variety of disorders of the central nervous system. Such disorders include mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalised anxiety disorders; schizophrenia and other psychotic disorders, for example, schizophreniform disorders, schizoaffective disorders, delusional disorders, brief psychotic disorders, shared psychotic disorders and psychotic disorders with delusions or hallucinations; delerium, dementia, and amnestic and other cognitive or neurodegenerative disorders, such as Alzheimer's disease, senile dementia, dementia of the Alzheimer's type, vascular dementia, and other dementias, for example, due to WV disease, head trauma, Parkinson's disease, Huntington's disease, Pick's disease, Creutzfeldt-Jakob disease, or due to multiple aetiologies; Parkinson's disease and other extra-pyramidal movement disorders such as medication-induced movement disorders, for example, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremour; substance-related disorders arising from the use of alcohol, amphetamines (or amphetamine-like substances), caffeine, cannabis, cocaine, hallucinogens, inhalants and aerosol propellants, nicotine, opioids, phenylglycidine derivatives, sedatives, hypnotics, and anxiolytics, which substance-related disorders include dependence and abuse, intoxication, withdrawal, intoxication delerium, withdrawal delerium, persisting dementia, psychotic disorders, mood disorders, anxiety disorders, sexual dysfunction and sleep disorders; epilepsy; Down's syndrome; demyelinating diseases such as MS and ALS and other neuropathological disorders such as peripheral neuropathy, for example diabetic and chemotherapy-induced neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid haemorrhage or cerebral oedema.

Tachykinin, and in particular substance P, activity is also involved in nociception and pain. The compounds of the present invention will therefore be of use in the prevention or treatment of diseases and conditions in which pain predominates, including soft tissue and peripheral damage, such as acute trauma, osteoarthritis, rheumatoid arthritis, musculo-skeletal pain, particularly after trauma, spinal pain, myofascial pain syndromes, headache, episiotomy pain, and burns; deep and visceral pain, such as heart pain, muscle pain, eye pain, orofacial pain, for example, odontalgia, abdominal pain, gynaecological pain, for example, dysmenorrhoea, and labour pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; ankylosing spondylitis, gout; and scar pain.

Tachykinin, and in particular substance P, antagonists may also be of use in the treatment of respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; inflammatory diseases such as inflammatory bowel disease, psoriasis, fibrositis, osteoarthritis, rheumatoid arthritis, pruritis and sunburn; allergies such as eczema and rhinitis; hypersensitivity disorders such as poison ivy; ophthalmic diseases such as conjunctivitis, vernal conjunctivitis, and the like; ophthalmic conditions associated with cell proliferation such as proliferative vitreoretinopathy; cutaneous diseases such as contact dermatitis, atopic dermatitis, urticaria, and other eczematoid dermatitis. Tachykinin, and in particular substance P, antagonists may also be of use in the treatment of neoplasms, including breast tumours, neuroganglioblastomas and small cell carcinomas such as small cell lung cancer.

Tachykinin, and in particular substance P, antagonists may also be of use in the treatment of gastrointestinal (GI) disorders, including inflammatory disorders and diseases of the GI tract such as gastritis, gastroduodenal ulcers, gastric carcinomas, gastric lymphomas, disorders associated with the neuronal control of viscera, ulcerative colitis, Crohn's disease, irritable bowel syndrome and emesis, including acute, delayed or anticipatory emesis such as emesis induced by chemotherapy, radiation, toxins, viral or bacterial infections, pregnancy, vestibular disorders, for example, motion sickness, vertigo, dizziness and Meniere's disease, surgery, migraine, variations in intercranial pressure, gastro-oesophageal reflux disease, acid indigestion, over indulgence in food or drink, acid stomach, waterbrash or regurgitation, heartburn, for example, episodic, nocturnal or meal-induced heartburn, and dyspepsia.

Tachykinin, and in particular substance P, antagonists may also be of use in the treatment of a variety of other conditions including stress related somatic disorders; reflex sympathetic dystrophy such as shoulder/hand syndrome; adverse immunological reactions such as rejection of transplanted tissues and disorders related to immune enhancement or suppression such as systemic lupus erythematosus; plasma extravasation resulting from cytokine chemotherapy, disorders of bladder function such as cystitis, bladder detrusor hyper-reflexia, frequent urination and urinary incontinence, including the prevention or treatment of overactive bladder with symptoms of urge urinary incontinence, urgency, and frequency; fibrosing and collagen diseases such as scleroderma and eosinophilic fascioliasis; disorders of blood flow caused by vasodilation and vasospastic diseases such as angina, vascular headache, migraine and Reynaud's disease; and pain or nociception attributable to or associated with any of the foregoing conditions, especially the transmission of pain in migraine. The compounds of the present invention are also of value in the treatment of a combination of the above conditions, in particular in the treatment of combined post-operative pain and post-operative nausea and vomiting.

The compounds of the present invention are particularly useful in the prevention or treatment of emesis, including acute, delayed or anticipatory emesis, such as emesis induced by chemotherapy, radiation, toxins, pregnancy, vestibular disorders, motion, surgery, migraine, and variations in intercranial pressure. For example, the compounds of the present invention are of use optionally in combination with other antiemetic agents for the prevention of acute and delayed nausea and vomiting associated with initial and repeat courses of moderate or highly emetogenic cancer chemotherapy, including high-dose cisplatin. Most especially, the compounds of the present invention are of use in the treatment of emesis induced by antineoplastic (cytotoxic) agents, including those routinely used in cancer chemotherapy, and emesis induced by other pharmacological agents, for example, rolipram. Examples of such chemotherapeutic agents include alkylating agents, for example, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; and cytotoxic antibiotics. Particular examples of chemotherapeutic agents are described, for instance, by D. J. Stewart in Nausea and Vomiting: Recent Research and Clinical Advances, Eds. J. Kucharczyk et al, CRC Press Inc., Boca Raton, Fla., USA (1991) pages 177-203, especially page 188. Commonly used chemotherapeutic agents include cisplatin, dacarbazine (DTIC), dactinomycin, mechlorethamine, streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil [R. J. Gralla et al in Cancer Treatment Reports (1984) 68(1), 163-172]. A further aspect of the present invention comprises the use of a compound of the present invention for achieving a chronobiologic (circadian rhythm phase-shifting) effect and alleviating circadian rhythm disorders in a mammal. The present invention is further directed to the use of a compound of the present invention for blocking the phase-shifting effects of light in a mammal.

The present invention is further directed to the use of a compound of the present invention or a pharmaceutically acceptable salt thereof, for enhancing or improving sleep quality as well as preventing and treating sleep disorders and sleep disturbances in a mammal. In particular, the present invention provides a method for enhancing or improving sleep quality by increasing sleep efficiency and augmenting sleep maintenance. In addition, the present invention provides a method for preventing and treating sleep disorders and sleep disturbances in a mammal which comprising the administration of a compound of the present invention or a pharmaceutically acceptable salt thereof. The present invention is useful for the treatment of sleep disorders, including Disorders of Initiating and Maintaining Sleep (insomnias) (“DIMS”) which can arise from psychophysiological causes, as a consequence of psychiatric disorders (particularly related to anxiety), from drugs and alcohol use and abuse (particularly during withdrawal stages), childhood onset DIMS, nocturnal myoclonus, fibromyalgia, muscle pain, sleep apnea and restless legs and non specific REM disturbances as seen in ageing.

The particularly preferred embodiments of the instant invention are the treatment of emesis, urinary incontinence, depression or anxiety by administration of the compounds of the present invention to a subject (human or companion animal) in need of such treatment.

The present invention is directed to a method for the manufacture of a medicament for antagonizing the effect of substance P at its receptor site or for the blockade of neurokinin-1 receptors in a mammal comprising combining a compound of the present invention with a pharmaceutical carrier or diluent. The present invention is further directed to a method for the manufacture of a medicament for the treatment of a physiological disorder associated with an excess of tachykinins in a mammal comprising combining a compound of the present invention with a pharmaceutical carrier or diluent.

The present invention also provides a method for the treatment or prevention of physiological disorders associated with an excess of tachykinins, especially substance P, which method comprises administration to a patient in need thereof of a tachykinin reducing amount of a compound of the present invention or a composition comprising a compound of the present invention. As used herein, the term “treatment” or “to treat” refers to the administration of the compounds of the present invention to reduce, ameliorate, or eliminate either the symptoms or underlying cause of the noted disease conditions, in a subject (human or animal) that suffers from that condition or displays clinical indicators thereof. The term “prevention” or “to prevent” refers to the administration of the compounds of the present invention to reduce, ameliorate, or eliminate the risk or likelihood of occurrence of the noted disease conditions, in a subject (human or animal) susceptible or predisposed to that condition.

The compounds of this invention are useful for antagonizing tachykinins, in particular substance P in the treatment of gastrointestinal disorders, central nervous system disorders, inflammatory diseases, pain or migraine and asthma in a mammal in need of such treatment. This activity can be demonstrated by the following assays.

Receptor Expression in COS: To express the cloned human neurokinin-1 receptor (NK1R) transiently in COS, the cDNA for the human NK1R was cloned into the expression vector pCDM9 which was derived from pCDM8 (INVITROGEN) by inserting the ampicillin resistance gene (nucleotide 1973 to 2964 from BLUESCRIPT SK+) into the Sac II site. Transfection of 20 ug of the plasmid DNA into 10 million COS cells was achieved by electroporation in 800 ul of transfection buffer (135 mM NaCl, 1.2 mM CaCl₂, 1.2 mM MgCl₂, 2.4 mM K₂HPO₄, 0.6 mM KH₂PO₄, 10 mM glucose, 10 mM BEPES pH 7.4) at 260 V and 950 uF using the MI [ GENEZAPPER (IBI, New Haven, Conn.). The cells were incubated in 10% fetal calf serum, 2 mM glutamine, 100 U/ml penicillin-streptomycin, and 90% DMEM media (GIBCO, Grand Island, N.Y.) in 5% CO₂ at 37° C. for three days before the assay.

Stable Expression in CHO: To establish a stable cell line expressing the cloned human NK1R, the cDNA was subcloned into the vector pRcCMV (INVITROGEN). Transfection of 20 ug of the plasmid DNA into CHO cells was achieved by electroporation in 800 ul of transfection buffer suplemented with 0.625 mg/ml Herring sperm DNA at 300 V and 950 uF using the EBI GENEZAPPER ( EBI). The transfected cells were incubated in CHO media [10% fetal calf serum, 100 U/ml pennicilin-streptomycin, 2 mM glutamine, 1/500 hypoxanthine-thymidine (ATCC), 90% E VIDM media (JRH BIOSCIENCES, Lenexa, Kans.), 0.7 mg/ml G418 (GIBCO)] in 5% CO₂ at 37° C. until colonies were visible. Each colony was separated and propagated. The cell clone with the highest number of human NK IR was selected for subsequent applications such as drug screening.

Assay Protocol using COS or CHO: The binding assay of human NK1R expressed in either COS or CHO cells is based on the use of ¹²⁵I-substance P (¹²⁵I-SP, from DU PONT, Boston, Mass.) as a radioactively labeled ligand which competes with unlabeled substance P or any other ligand for binding to the human NK1R. Monolayer cell cultures of COS or CHO were dissociated by the non- enzymatic solution (SPECIALTY MEDIA, Lavallette, N.J.) and resuspended in appropriate volume of the binding buffer (50 mM Tris pH 7.5, 5 mM MnCl₂, 150 mM NaCl, 0.04 mg/ml bacitracin, 0.004 mg/ml leupeptin, 0.2 mg/ml BSA, 0.01 mM phosphoramidon) such that 200 ul of the cell suspension would give rise to about 10,000 cpm of specific ¹²⁵I-SP binding (approximately 50,000 to 200,000 cells). In the binding assay, 200 ul of cells were added to a tube containing 20 ul of 1.5 to 2.5 nM of ¹²⁵I-SP and 20 ul of unlabeled substance P or any other test compound. The tubes were incubated at 4° C. or at room temperature for 1 hour with gentle shaking. The bound radioactivity was separated from unbound radioactivity by GF/C filter (BRANDEL, Gaithersburg, Md.) which was pre-wetted with 0.1% polyethylenimine. The filter was washed with 3 ml of wash buffer (50 mM Tris pH 7.5, 5 mM MnCl₂, 150 mM NaCl) three times and its radioactivity was determined by gamma counter. The activation of phospholipase C by NK1R may also be measured in CHO cells expressing the human NK1R by determining the accumulation of inositol monophosphate which is a degradation product of IP₃. CHO cells are seeded in 12-well plate at 250,000 cells per well. After incubating in CHO media for 4 days, cells are loaded with 0.025 uCi/ml of ³H-myoinositol by overnight incubation. The extracellular radioactivity is removed by washing with phosphate buffered saline. LiCl is added to the well at final concentration of 0.1 mM with or without the test compound, and incubation is continued at 37° C. for 15 min. Substance P is added to the well at final concentration of 0.3 nM to activate the human NK1R. After 30 min of incubation at 37° C., the media is removed and 0.1 N HCl is added. Each well is sonicated at 4° C. and extracted with CHCl₃/methanol (1:1). The aqueous phase is applied to a 1 ml Dowex AG 1X8 ion exchange column. The column is washed with 0.1 N formic acid followed by 0.025 M ammonium formate-0.1 N formic acid. The inositol monophosphate is eluted with 0.2 M ammonium formate-0.1 N formic acid and quantitated by beta counter. In particular, the intrinsic tachykinin receptor antagonist activities of the compounds of the present invention may be demonstrated by these assays. The compounds of the following examples have activity in the aforementioned assays in the range of 0.05 nM to 10 μM. The activity of the present compounds may also be demonstrated by the assay disclosed by Lei, et al., British J. Pharmacol., 105, 261-262 (1992).

According to a further or alternative aspect, the present invention provides a compound of the present invention for use as a composition that may be administered to a subject in need of a reduction of the amount of tachykinin or substance P in their body.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The compositions containing compounds of the present invention may be presented in unit dosage form and may be prepared by any of the methods well known in the art of pharmacy. The term “unit dosage form” is taken to mean a single dose wherein all active and inactive ingredients are combined in a suitable system, such that the patient or person adminstering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples in the pharmacy arts of unit dosage forms. The compositions containing compounds of the present invention may also be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound of the invention to the individual in need of treatment in a form that can be introduced into that individuals body in a therapeutically useful form and therapeutically effective amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories. The term “therapeutically effective amount” refers to a sufficient quantity of the compounds of the present invention, in a suitable composition, and in a suitable dosage form to treat or prevent the noted disease conditions.

The compounds of the present invention may be administered in combination with another substance that has a complimentary effect to the tachykinin and substance P inhibitors of the present invention. Accordingly, in the prevention or treatment of emesis, a compound of the present invention may be used in conjunction with other anti-emetic agents, especially 5HT₃ receptor antagonists, such as ondansetron, granisetron, tropisetron, palenosetron and zatisetron, a corticosteroid, such as dexamethasone, or GABA_{B} receptor agonists, such as baclofen. Likewise, for the prevention or treatment of migraine a compound of the present invention may be used in conjunction with other anti- migraine agents, such as ergotamines or 5HT₁ agonists, especially sumatriptan, naratriptan, zolmatriptan or rizatriptan.

It will be appreciated that for the treatment of depression or anxiety, a compound of the present invention may be used in conjunction with other anti-depressant or anti-anxiety agents, such as norepinephrine reuptake inhibitors, selective serotonin reuptake inhibitors (SSRIs), monoamine oxidase inhibitors (MAOIs), reversible inhibitors of monoamine oxidase (R hIAs), serotonin and noradrenaline reuptake inhibitors (SNRls), α-adrenoreceptor antagonists, atypical anti-depressants, benzodiazepines, ⁵-HT_{1A} agonists or antagonists, especially 5-HT_{1A} partial agonists, corticotropin releasing factor (CRF) antagonists, and pharmaceutically acceptable salts thereof. For the treatment or prevention of eating disorders, including obesity, bulimia nervosa and compulsive eating disorders, a compound of the present invention may be used in conjunction with other anorectic agents. It will be appreciated that for the treatment or prevention of pain or nociception or inflammatory diseases, a compound of the present invention may be used in conjunction with an antiinflaim matory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent.

It will be appreciated that when using any combination described herein, both the compound of the present invention and the other active agent(s) will be administered to a patient, within a reasonable period of time. The compounds may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, one active component may be administered as a tablet and then, within a reasonable period of time, the second active component may be administered either as an oral dosage form such as a tablet or a fast-dissolving oral dosage form. By a “fast dissolving oral formulation” is meant, an oral delivery form which when placed on the tongue of a patient, dissolves within about 10 seconds. By “reasonable period of time” is meant a time period that is not in excess of about 1 hour. That is, for example, if the first active component is provided as a tablet, then within one hour, the second active component should be administered, either in the same type of dosage form, or another dosage form which provides effective delivery of the medicament.

The compounds of this invention may be administered to patients (humans and animals, including companion animals, such as dogs, cats and horses) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician.

In the treatment of the conditions associated with an excess of tachykinins, a suitable dosage level of the compounds of the present invention, or pharmaceutically acceptable salts thereof, is about 0.001 to 50 mg/kg per day, in particular about 0.01 to about 25 mg/kg, such as from about 0.05 to about 10 mg/kg per day. The dosage range will generally be about 0.5 to 1000 mg per patient per day, which may be administered in single or multiple doses. Preferably, the dosage range will be about 0.5 mg to 500 mg per patient per day; more preferably about 0.5 mg to 200 mg per patient per day; and even more preferably about 5 mg to 50 mg per patient per day. Specific dosages of the compounds of the present invention, or pharmaceutically acceptable salts thereof, for administration include 1 mg, 5 mg, 10 mg, 30 mg, 100 mg, and 500 mg. Pharmaceutical compositions of the present invention may be provided in a formulation comprising about 0.5 mg to 1000 mg active ingredient; more preferably comprising about 0.5 mg to 500 mg active ingredient; or 0.5 mg to 250 mg active ingredient; or 1 mg to 100 mg active ingredient. Specific pharmaceutical compositions for treatment or prevention of excess tachykinins comprise about 1 mg, 5 mg, 10 mg, 30 mg, 100 mg, and 500 mg of active ingredient.

Several methods for preparing the compounds of this invention are illustrated in the following Examples. Starting materials and the requisite intermediates are in some cases commercially available, or can be prepared according to literature procedures or as illustrated herein. All NMR spectra were obtained on instrumentation at a field strength of 400 or 500 MHz in either CDCl₃ or CD₃OD with reported chemical shifts as δ. The UPLC/MS analyses were obtained using an Agilent 1100 Series BPLC in combination with a Waters Micromass ZQ mass spectrometer. The HPLC RP column was a Waters Exterra MS-C18 (5 μm) 3.0×50 mm column eluting with a 10-100% acetonitrile/water (both containing 0.05% TFA) gradient over 3.75 min with a run time of 5.50 min. UV monitoring was done at 210 nM. Retention times (Rt) are reported in minutes based on the MS data. The reported m/e value was usually the parent molecular ion, except when the 100% ion was not the parent ion as also indicated. Preparative chiral HPLC was done with the indicated Chiracel 25×250 mm columns eluting at 9 mL per min with the indicated percent isopropanol/heptanes solvent mixture. Retention times (Rt) are reported in minutes based on the UV chromatograin monitored at 210 or 254 nm.

### EXAMPLE 1

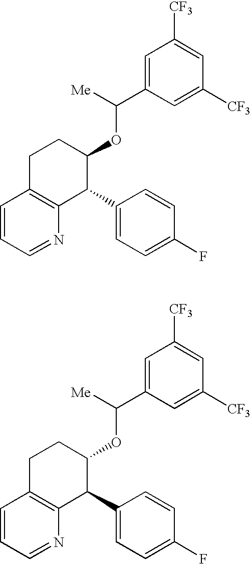

### Racemic (7,8-trans)-7-{1-[3,5-bis(Trifluoromethyl)phenyl]ethoxy}-8-(4-fluorophenyl)-5,6,7,8-tetrahydroquinoline

Step A: 8-Benzvlidene-5,6,7,8-tetrah vdroquinoline

A mixture of 4.876 g (36.6 mmol) of 5,6,7,8-tetrahydroquinoline, 5.83g (54.9 mmol) benzaldehyde and 6.58 mL (69.6 nunol) of acetic anhydride was heated at 170° C. for 20 hr. The resulting mixture was cooled to RT and volatiles distilled under vacuum. The residue was diluted with ˜20 mL MeOH and 5 mL of %N aq. NaOH was added. The resulting mixture was stirred at RT for 2 hr then diluted with ether. The layers were separated and the ether layer was wadhed with aq. NaOh, brine, dried over drying agent and filtered. The solvent was removed under vacuum and the residue was purified by column chromatography on silica gel eluting with hexanes/EtOAc (1/10 to 9/1) to afford the title compound as a light yellow solid. ¹H-NMR (CDCl₃): δ8.52 (d of d, 1HJ=1.6, 4.5 Hz), 8.02 (s, 1H), 7.48 (d, 2H, J=7.5Hz), 7.39-7.44 (m, 3H), 7.29 (t, 1H, J=6.1 Hz), 7.11 (d of d, IH, J=4.6, 2.9 Hz), 2.93-2.95 (m, 2H), 2.88 (t, 2H, J=6.2 Hz), 1.86-1.91 (m, 2H), ppm. MS: 222 (MH)⁺

Step B: 6,7-Dihydroquinolin-8(5H)-one

A solution of 6.23 g (28.15 mmol) of the intermediate of step A in ˜100 mL methanol and 20 mL methylene chloride, cooled in a dry ice/acetonitrile bath, was treated with ozone until the solution became blue in color. The excess ozone was removed by a nitrogen stream and 5 mL dimethylsulfide was added. The cooling bath was removed and the reaction mixture was stirred at RT for 1 hr. The solvent was evaporated under vacuum. The residue was purified by flash column chromatography on silica gel eluting with EtOAc/hexanes/2N NH₃ in MeOH (5/5/1) to give the title compound. ¹H-NMR (CDCl₃): δ8.68 (d of d, 1H, J=1.3, 4.3 Hz), 7.66 (d, 1H, J=5.7 Hz), 7.38 (d of d, 1H, J=4.3, 7.7 Hz), 3.02 (t, 2H, J=6.2 Hz), 2.79 (6.1 Hz), 2.16-2.19 (m, 2H) :ppm. MS: 148 (MH)⁺.

Step C: 8-(4-Fluorophenyl)-5,6,7,8-tetrahvdroquinolin-8-ol

To a solution of 4.14 g (28.1 mmol) of 6,7-dihydroquinolin-8(5H)-one (step B) in dry THF, under nitrogen atmosphere at 0 ° C. was added dropwise 42.2mL (1.5equiv.) of a 1.0 M solution of 4-fluorophenylmagnesium bromide in THF. The cooling bath was removed and the reaction mixture was stirred at RT for 2 hr. The resulting mixture was diluted with methylene chloride and quenched with excess water. The layers were separated and the organic layer dried over Na₂SO₄ drying agent, filtered through filter aid and the solvent evaporated under vacuum to afford the title compound which was used without further purification. 8: ¹H-NMR (CDC13): 8.47 (d, 1H, 3.9 Hz), 7.54 (d, 1H, J=7.8 Hz), 7.21 (d of d, 1H, J=5.6, 7.8 Hz), 7.09-7.12 (, 2H), 6.96 (t, 2H, J=8.7 Hz), 4.31 (S, 1H), 2.90-2.95 (m, 2H), 2.24-2.27 (m, 2H), 1.86-1.93 (m, 1H), 1.62-1.71 (m, 1H). MS: (MH)⁺, 244.

Step D: 8-(4-Fluorophenvl)-5,6-dihydroquirioline

To a solution of 2.32 g (9.55 mmol) 8-(4-Fluorophenyl)-5,6,7,8-tetrahydroquinolin-8-ol (step C) in 30 mL toluene under nitrogen atmosphere was added 2.48 mL (38.2 mmol) methanesulfonic acid. The reaction mixture was heated at reflux for 2 hr. The reaction mixture was cooled to RT, diluted with ether and transferred to a separatory funnel. The organic layer was washed with 5 N aq. NaOH, brine, dried over anhydrous sodium sulfate, filtered and the solvent evaporated under vacuum to afford the title compound as a light yellow solid. ¹H-NMR (CDCl₃): δ8.44 (d of d, 1H, J=1.4, 4.8 Hz), 7.50 (d, 1H, J=8.0 Hz), 7.45 (d of d, 2H, J=5.4, 8.6 Hz), 7.08-7.11 (m, 3H), 6.44 (t, 1H, J=4.8 Hz), 2.94 (t, 2H, J=8.0 Hz), 2.48-2.52 (m, 2H). MS: 226 (MH)⁺.

Step E: Racemic (7,8-trans )-8-(4-Fluorophenyl)-5,6,7,8-tetrahydroquinolin-7-ol

A solution of 1.69 mL (14.2 mmol) of 2,3-dimethyl-2-butene in 15 mL dry THF under nitrogen atmosphere at 0° C. was treated with 7.1 mL (14.2 mmol)of a 2.0 M solution of borane-dimethylsulfide complex in THF. The resulting mixture was stirred at RT for 1 hr at which time was added a solution of 0.8 g (3.55 mmol) 8-(4-fluorophenyl)-5,6-dihydroquinoline (step D) in 5 mL THF. The resulting mixture was heated at 66° C for 7hr. The reaction mixture was cooled in an ice bath and carefully treated with 7.5 mL of 30% aq. H₂O₂ and 7.5 mL 5 N aq. NaOH. After stirring vigorously for 10 hours, the reaction mixture was extracted with EtOAc, dried over drying agent and the solvent evaporated under vacuum. The residue was purified by colu mn n chromatography on silica gel eluting with hexanes/EtOAc/2N NH₃ in MeOH (10/10/1) to afford the title compound. ¹H-NMR (CDCl₃): δ: 8.38 (D, 1h, J=4.6 Hz), 7.51 (d, 1H,J =7.7 Hz), 7.12 (d of d, 1H, J=3.6, 7.6 Hz), 6.95-7.02 (m, 4H), 4.20 (d, 1H, J=6.4 Hz)4.09-4.12 (m, 2H), 2.90-3.06 (m, 2H), 2.63 (bs, 1H), 2.07-2.12 (m, 1H), 1.86-1.93 (m, 1H). LC-MS: 244 (MH)⁺.

Step F: Racemic (7,8-trans)-8-(4-fluorophenyl)-5,6,7,8-tetrahydroquinolin-7-yl 3,5-bis(trifluoromethvl)benzoate

To a solution of 0.32g (1.32 mmol) of racemic (7,8-trans)-8-(4-Fluorophenyl)-5,6,7,8-tetrahydroquinolin-7-ol (step E) in 15 mL dry methylene chloride under nitrogen atmosphere at RT was added 0.032 g (0.26 mmol) DMAP, 0.37 mL (2.64 mmol) TEA and 0.36 mL (1.97 mmol) of 3,5-bis(trifluoromethyl)benzoyl chloride. The resulting mixture was stirred at RT for 3 hr then diluted with ether. The reaction mixture was transferred to a separatory funnel, washed with sat. aq. NaHCO₃ and then brine. The combined organic layers dried over sodium sulfate, filtered and the solvent evaporated under vacuum. The residue was purified by prep TLC eluting with EtOAc/hexanes (1/6) to afford the title compound as a white foam. ¹H-NMR (CDCl₃): δ: 8.51 (d, 1H, J=4.6 Hz), 8.41 (s, 2H), 8.01 (s, 1H), 7.63 (d, 1H, J=7.6 Hz), 7.23 (d of d, 1H, J=4.8, 7.8 Hz), 7.09 (d of d, 2H, J5.3, 8.5 Hz), 7.03 (t, 2H, J=8.4 Hz), 5.59-5.62 (m, 1H), 4.65 (d, 1H, J=4.6 Hz), 3.07-3.20 (m, 2H), 2.15-2.31 (m, 2H). LC-MS: 484 (MH)⁺.

Step G: Racemic (7,8-trians)-7-({ 1-[3,5-bis(trifluoromethyl)phenyl]vinyl}oxy)-8-(4-fluorophenyl)-5,6,7,8-tetrahydroquinoline

To a solution of 0.60 g (1.24 mmol) of racemic (7,8-trans)-8-(4-fluorophenyl)-5,6,7,8-tetrahydroquinolin-7-yl 3,5-bis(trifluoromethyl)benzoate (step F) in 10 mL dry THF under nitrogen atmosphere at 0° C. was added 5 mL (2.48 mmol) of a 0.5 M solution of Tebbe reagent in toluene. The resulting mixture was stirred at 0° C. for 0.5 hr then carefully quenched by the dropwise addition of 5 mL of 5.0 N aq. NaOH, then 5 mL water. The resulting suspension was stirred vigorously for 2 hr then diluted with ether. The organic layer was separated and washed with brine. The combined aq. layers were back extracted with ether. All the organic layers were dried over sodium sulfate filtered and the solvent was evaporated under vacuum. The residue was purified by prep TLC eluting with EtOAc/hexanes (1/6) to afford the title compound as a yellow solid. ¹H-NMR (CDCl ₃): δ: 8.49 (d of d, H, J=1.4,4.6Hz), 7.87 (s, 2H), 7.81 (s, 1H), 7.61 (d, 1H, J=7.8Hz), 7.31 (t, 2H, J=5.7Hz), 7.19-7.24 (m, 3H), 5.02 (d, 1H, J=3.4 Hz), 4.75-4.77 (m, 1H), 4.70 (d, 1H, J=3.7 Hz), 4.62 (d, 1H, J=4.1 Hz), 2.99-3.18 (m, 2H), 2.18-2.28 (m, 2H). LC-MS: 482(MH)⁺.

Step H: Racemic (7,8-trans)-7-{1-[3,5-bis(trifluoromethyl)phenyl]ethoxy}-8-(4-fluorophenyl)-5,6,7,8-tetrahydroquinoline

A solution of 0.50 g racemic (7,8-trans)-7-({1-[3,5-bis(trifluoromethyl)phenyl]vinyl}-oxy)-8-(4-fluorophenyl)-5,6,7,8-tetrahydroquinoline (step G) in 50 mL EtOH and 5 mL EtOAc was hydrogenated at 50 PSI hydrogen over 0.5g of 10% Pd-C for 6 hr at RT. The catalyst was filtered through filter aid and the solvent of the filtrate was evaporated under vacuum. The residue was redissolved in 50 mL EtOH and 5 mL EtOAc and was hydrogenated at 50 PSI hydrogen over 0.5g of 10% Pd-C for 16 hr at RT. The catalyst was filtered through filter aid and the solvent of the filtrate was evaporated under vacuum to give the crude title compounds which were purified by prep TLC eluting with EtOAc/CH₂Cl₂ (1/20) to afford the two diastereomers. The less polar isomer, ¹H-NM k (CDCl₃): δ: 8.41 (d of d, 1H, J=1.6, 4.6 Hz), 7.77 (s, 1H), 7.62 (s, 2H), 7.52 (d, 1H, J=7.7 Hz), 7.12 (d of d, 1H, J=4.8, 7.8 Hz), 6.89 (t, 2H, J=8.6 Hz), 6.79-6.83 (m, 2H), 4.76 (q, 1H, J=6.4 Hz), 4.31 (d, 1H, J=5.0Hz), 3.74-3.77 (m, 1H), 3.06-3.12 (m, 1H), 2.86-2.91 (m, 1H), 2.07-2.13 (m, 1H), 1.93-1.99 (m, 1H), 1.48 (d, 3H, J=6.4 Hz). LC-MS: 484 (MH)⁺. The more polar isomer, ¹H-NMR (CDCl₃): δ: 8.46 (d of d, 1H, J=1.6 Hz), 7.81 (s,, 1H), 7.78 (s, 2H), 7.51 (d, 1H, J=7.5 Hz). 7.14 (d of d, 1H, J=4.6, 7.5 Hz), 6.94-7.01 (m, 4H), 4.64 (q, 1H, J=6.6 Hz), 4.45 (d, 1H, J=5.1 Hz), 3.74-3.77 (m, 1H), 2.99-3.05 (m, 1H), 2.80-2.85 (m, 1H), 1.88-1.93 (m, 1H), 1.78-1.84 (m, 1H), 1.32 (d, 3H, J=6.4 Hz). LC-MS: 484 (MH)⁺.

### EXAMPLE 2

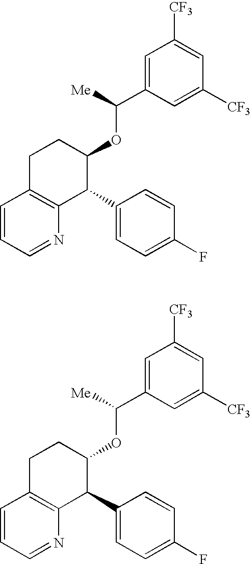 (7R,8R)-7-{(1S)- 1-[3,5-bis(Trifluoromethyl)phenyl]ethoxy}-8-(4-fluorophenyl)-5,6,7,8-tetrahydroquinoline and (7S,8S)-7-{(1R)-1-[3,5-bis(trifluoromethyl)phenyl]ethoxy}-8-(4-fluorophenyl)- 5,6,7,8-tetrahydroquinoline

Starting with 0.17 g of the racemic mixture of the less polar diastereomer of product of Example 1, step H was separated by chiral HPLC using CHIRACEL AD column eluting with heptane/i-PrOH (85/15) to afford 66.5 mg of the first eluting isomer (7R,8R)-7-{(1S)-1-[3,5-bis(trifluoromethyl)phenyl]ethoxy} -8-(4-fluorophenyl)-5,6,7,8-tetrahydroquinoline (Rₜ=10.35 min) and 60 mg of the second eluting isomer (7S,8S)-7-{(1R)-1-[3,5-bis(trifluoromethyl)phenyl]ethoxy}-8-(4-fluorophenyl)-5,6,7,8-tetrahydroquinoline (Rₜ=13.33 min). ¹H-NMR (CDC13): δ: 8.41 (d of d, 1H, J=1.6, 4.6 Hz), 7.77 (s, 1H), 7.62 (s, 2H), 7.52 (d, 1H, J=7.7 Hz), 7.12 (d of d, 1H, J=4.8, 7.8 Hz), 6.89 (t, 2H, J=8.6 Hz), 6.79-6.83 (m, 2H), 4.76 (q, 1H, J=6.4 Hz), 4.31 (d, 1H, J=5.0Hz), 3.74-3.77 (m, 1H), 3.06-3.12 (m, 1H), 2.86-2.91 (m, 1H), 2.07-2.13 (m, 1H), 1.93-1.99 (m, 1H), 1.48 (d, 3H, J=6.4 Hz). LC-MS: 484 (MH)⁺.

### EXAMPLE 3

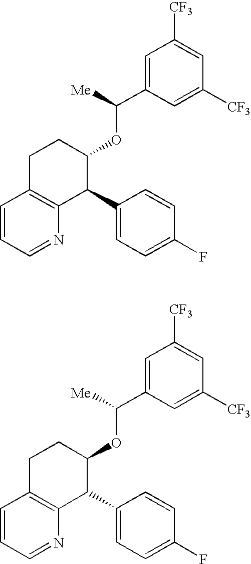 (7R,8R)-7-{(1R)-1-[3,5-bis(Trifluoromethyl)phenyl]ethoxy}-8-(4-fluorophenyl)-5,6,7,8-tetrahydroquinoline and (7S,8S)-7-{(1S)-1-[3,5-bis(trifluoromethyl)phenyl]ethoxy}-8-(4-fluorophenyl)-5,6,7,8-tetrahydroquinoline

Starting with 0.21 g of the racemic mixture of the more polar diastereomer of product of Example 1, step H was separated by chiral HPLC using CHIRACEL AD column eluting with hexanea/EtOH (95/5) to afford the first eluting isomer (7R,8R)-7-{(1R)-1-[3,5-bis(trifluoromethyl)phenyl]ethoxy}-8-(4-fluorophenyl)-5,6,7,8-tetrahydroquinoline (Rₜ=6.84 min) and the second eluting isomer (7S,8S)-7-{(1 S)-1-[3,5-bis(trifluoromethyl)phenyl]ethoxy}-8-(4-fluorophenyl)-5,6,7,8-tetrahydroquinoline (Rₜ=9.31 min). ¹H-NMR (CDCl₃): δ: 8.46 (d of d, 1H, J=1.6 Hz), 7.81 (s, 1H), 7.78 (s, 2H), 7.51 (d, 1H, J=7.5 Hz). 7.14 (d of d, 1H, J=4.6, 7.5 Hz), 6.94-7.01 (m, 4H), 4.64 (q, 1H, J=6.6 Hz), 4.45 (d, 1H, J=5.1 Hz), 3.74-3.77 (m, 1H), 2.99-3.05 (m, 1H), 2.80-2.85 (m, 1H), 1.88-1.93 (m, 1H), 1.78-1.84 (m, 1H), 1.32 (d, 3H, J=6.4 Hz). LC-MS: 484(MH)⁺.

### EXAMPLE 4

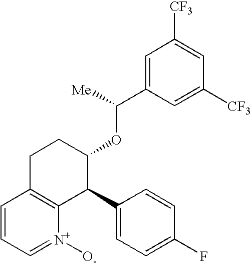 (7S,8S)-7-{(1R)-1-[3,5-bis(trifluoromethyl)phenyl]ethoxy}-8-(4-fluorophenyl)-5,6,7,8-tetrahydroquinoline 1-oxide

To a solution of 49.4 mg (0.1 mmol) of (7S,8S)-7-{(1R)-1-[3,5-bis(trifluoromethyl)-phenyl]ethoxy}-8-(4-fluorophenyl)-5,6,7,8-tetrahydroquinoline (second eluting isomer of Example 2, step A) in 3 mL dry CH₂Cl₂ was added 69mg (0.31 mmol) 3 equiv. of 77% m-CPBA. The resulting mixture was stirred at RT for 16 hr then 2 mL of 2 N aq. NaOH was added. The resulting mixture was stirred at RT for 0.5 hr then was extracted with methylene chloride. The combined extracts were washed with brine, dried over drying agent, filtered and the solvent was evaporated under vacuum to afford the title compound as a solid. ¹H-NMR (CDCl₃): δ: ppm.8.18 (bs, 1H), 7.83 (s, 1H), 7.78 (s, 2H), 7.22-7.23 (m, 2H), 6.94 (t, 2H, J=8.4 Hz), 6.84-6.87 (m, 2H), 4.84 (q, 1H, J=6.6 Hz), 4.74 (bs, 1H), 3.85-3.87 (m, 1H), 3.12-3.20 (m, 1H), 2.90-2.95 (m, 1H), 2.00-2.05 (m, 1H), 1.82-1.89 (m 1H), 1.50 (d, 3H, J=6.4 Hz). MS: 500 (MH)⁺.

### EXAMPLE 5

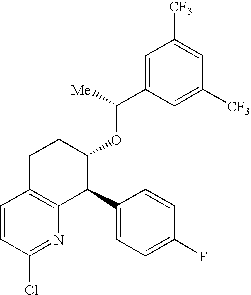 (7S,8S)-7-{(1R)-1-[3,5-bis(Trifluoromethyl)phenyl]ethoxy}-2-chloro-8-(4-fluorophenyl)-5,6,7,8-tetrahydroquinoline

A solution of 25 mg (0.05 mmol) of (7S,8S)-7-{(1R)-1-[3,5-bis(trifluoromethyl)-phenyl]ethoxy3-8-(4-fluorophenyl)-5,6,7,8-tetrahydroquinoline 1-oxide (Example 4) in 3 mL POCI₃ was heated at 120° C. for 2.5 hr. The reaction mixture was cooled to RT and the solvent removed under vacuum. The residue was dissolved in methylene chloride and washed with sat. aq. NaHCO₃. The aqueous layer was extracted with methylene chloride. The combined extracts were washed with brine, dried over drying agent, filtered through filter aid and the solvent was evaporated under vacuum. The residue was purified by prep TLC eluting with EtOAc/hexanes (1/5) to afford the title compound. ¹H-NMR (CDCl₃): δ: 7.81 (s, 1H), 7.69 (s, 2H), 7.51 (d, 1H, J=8.0 Hz), 7.19 (d, 1H, J=6.0 Hz), 6.92 (t, 2H, J=8.7 Hz), 6.74-6.78 (m, 2H), 4.78 (q, 1H, J=6.7 Hz), 4.31 (d, 1H, J=3.9 Hz), 3.77-3.80 (m, IH),3.05-3.12 (m, 1H), 2.83-2.86 (m, 1H), 1.94-1.97(m,2H), 1.499 (d, 3H, J=6.6 Hz). LC-MS: 518 (MH)⁺.

### EXAMPLE 6

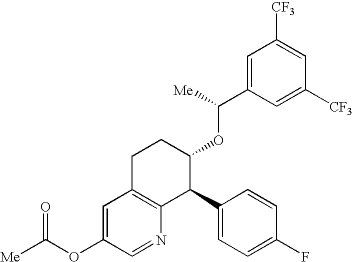 (7S,8S)-7-{(1R)-1-[3,5-bis(Trifluoromethyl)phenyl]ethoxy}-8-(4-fluorophenyl)-5,6,7,8-tetrahydroquinolin-3-yl acetate

A solution of 30.8 mg (0.062 mmol) of (7S,8S)-7-{(1R)-1-[3,5-bis(trifluoromethyl)-phenyl]ethoxy}-2-chloro-8-(4-fluorophenyl)-5,6,7,8-tetrahydroquinoline (Example 5) in 4 mL acetic anhydride was heated at 120° C. for 1.5 hr. The reaction mixture was cooled to RT and the solvent removed under vacuum. The residue was dissolved in MeOH and stirred for 0.5 hr. The solvent was evaporated under vacuum and the residue was purified by reverse phase prep HPLC (CH₃CN/water) to afford the title compound. ¹H-NMR (CDCl₃): δ: 8.43 (d, 1H, J=2.6 Hz), 7.81 (s, 1H), 7.76 (d, 1H, J=2.6 Hz), 7.71 (s, 2H), 7.07-7.09 (m, 4H), 4.88 (d, 1H, J=4.6 Hz), 4.50 (q, 1H, J=6.4 Hz), 3.92-33.96 (m, 1H), 3.20-3.27 (m, 1H), 2.88-2.94 (m, 1H), 2.40 (s, 3H), 1.82-1.99 (m, 2H). LC-MS: 541 (MH)⁺. Also isolated was (7S)-7-{(1R)-1-[3 ,5-bis(trifluoromethyl)phenyl]ethoxy}-8-(4-fluorophenyl)-5,6,7,8-tetrahydroquinolin-8-yl acetate; LC-MS: 564 (M+Na)⁺.

### EXAMPLE 7

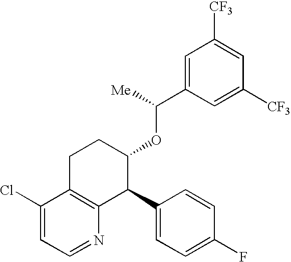 (7S,8S)-7-{(1R)-1-[3,5-bis(Trifluoromethyl)phenyl]ethoxy}-4-chloro-8-(4-fluorophenyl)-5,6,7,8-tetrahydroquinoline

A solution of 247 mg (0.49 mmol) of (7S,8S)-7-{(1R)-1-[3,5-bis(trifluoromethyl)-phenyl]ethoxy}-8-(4-fluorophenyl)-5,6,7,8-tetrahydroquinoline 1-oxide (Example 4) in 3 mL POCI₃ was stirred at RT for 0.5 hr. The solvent removed under vacuum. The residue was dissolved in methylene chloride and washed with sat. aq. NaHCO₃. The aqueous layer was extracted with methylene chloride (2×). The combined extracts were dried over drying agent, filtered and the solvent was evaporated under vacuum. The residue was dissolved in methylene chloride and equal amount 2 N aq. NaOH. The mixture was stirred at RT for 36 hr. The layers were separated and the aqueous layer was extracted with methylene chloride (2×). The combined extracts were dried over drying agent, filtered and the solvent was evaporated under vacuum. The residue was purified by reverse phase prep BPLC (CH₃CN/water) to afford the title compound. ¹H-NMR (CDCl₃): δ: 8.52 (d, 1H, J=6.2 Hz), 7.80 (s, 1H), 7.76 (d, 1H, J=6.1 Hz), 7.67 (s, 2H), 6.95 (t, 2H, J=8.7 Hz), 6.76-6.79 (m, 2H), 4.86 (q, 1H, J=6.4 Hz), 4.82 (bs, 1H), 3.87-3.89 (m, 1H), 3.12-3.15 (m, 1H), 2.02-2.21 (m, 2H), 1.49 (d, 3H, J=7.6 Hz). LC-MS: 518 (MH)⁺. Also isolated was starting materials (66 mg) and the 2-chloro isomer (27 mg).

### EXAMPLE 8

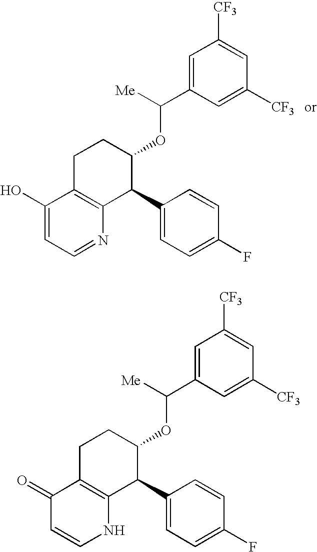 Racemic (7,8-trans)-7-{1-[3,5-bis(trifluoromethyl)phenyl]ethoxy}-8-(4-fluorophenyl)-5,6,7,8-tetrahydroquinolin-4-ol or racemic (7,8-trans)-7-{ 1-[3,5-bis(trifluoromethyl)phenyl]ethoxy}-8-(4-fluorophenyl)-5,6,7,8-tetrahydroquinolin-4(1H)-one
Step A: Racemic (7,8-trans)-8-(4-Fluorophenyl)-5,6,7,8-tetrahydroquinolin-7-ol 1-oxide

The title compound was prepared from racemic (7,8-trans)-8-(4-fluorophenyl)-5,6,7,8-tetrahydroquinolin-7-ol (Example 1, step E) according to the procedure of Example 4 to afford the title compound. ¹H-NMR (CDCl₃): δ: 8.11 (d, 1H, J=6.2 Hz), 7.23 (d, 1H, J=7.7 Hz), 7.17 (d of d, 1H, J=2.1, 6.6 Hz), 7.00 (t, 2H, J=8.7 Hz), 6.94-6.97 (m, 2H), 5.06 (s,1H), 4.48 (bs, 1H), 4.31-4.33 (m, 1H), 3.14-3.21 (m, 1H), 2.84-2.89 (m, 1H), 1.75-1.92 (m, 2H). LC-MS: 260 (MH)⁺.

Step B: 4-Chloro-8-(4-fluorophenvl)-5,6-dihydroquinoline

A solution of 0.56 g (2.16 mmol) of racemic (7,8-trans)-8-(4-Fluorophenyl)-5,6,7,8-tetrahydroquinolin-7-ol 1-oxide (step A) in 20 mL POCl₃ was heated at 80° C. for 2 hr. The solvent removed under vacuum. The residue was cooled to 0° C. dissolved 2 N ammonia in MeOH. The resulting mixture was stirred at RT for 16 hr. The mixture was poured into ether and washed with brine. The organic layer was dried over drying agent, filtered and the solvent was evaporated under vacuumn. The residue was purified by prep TLC eluting with EtOAc/hexanes (1/45) to afford the title compound. ¹H-NMR (CDCl₃): δ: 8.32 (d, 1H, J=5.3 Hz), 7.42 (d of d, 2H, J=5.5, 8.7 Hz), 7.20 (d, 1H, J=5.3 Hz), 7.11 (t, 2H, J=8.7 Hz), 6.48 (t, 1H, J=4.6 Hz), 3.11 (t, 2H, J=8.2 Hz), 2.52-2.57 (m, 2H). LC-MS: 260 (MH)⁺.

Step C: 8-(4-Fluorophenyl)-4-methox U-5,6-dihydroquinoline

A solution of 0.27 g (1.05 mmol) of 4-chloro-8-(4-fluorophenyl)-5,6-dihydroquinoline (step B) in 5 mL MeOH was added 4 mL of a solution of 25 wt % NaOMe in MeOH. The resulting slightly red mixture was heated at 95° C. for 58 hr. The mixture was cooled to RT then poured into sat. aq. NaHCO₃. The aqueous layer was extracted with EtOAc. The combined extracts were washed with brine, dried over drying agent, filtered and the solvent was evaporated under vacuum. The residue was purified by silica gel column chromatography eluting with EtOAc/hexanes (1/8 to 1/4) to afford of the title compound. ¹H-NM k (CDCl₃): δ: 8.36 (d, 1H, J=5.7 Hz), 7.43 (d of d, 2H, J=5.7, 8.9 Hz), 7.10 (t, 2H, J=8.9 Hz), 6.73 (d, 1H, J=5.5 Hz), 6.41 (t, 1H, J=4.8 Hz), 3.94 (s, 3H), 2.93 (t. 2H, J=8.3 Hz), 2.44-2.48 (m, 2H). LC-MS: 256 (MH)⁺.

Step D: Racemic (7,8-trans )-8-(4-fluorophenyl )-4-methoxy-5,6,7,8-tetrahydroquinolin-7

The title compound was prepared from 8-(4-fluorophenyl)-4-methoxy-5,6-dihydroquinoline (step C) according to the procedure of Example 1, step E. ¹H-NMR (CDCl ₃): δ: 8.30 (d, 1H, J=5.5 Hz), 6.94-6.99 (m, 4H), 6.66 (d, 1H, J=5.5 Hz), 4.15 (d, 1H,, J=5.9 Hz), 4.05 (m, 1H), 3.92 (s, 3H), 2.90-2.95 (m, 1H), 2.02-2.06 (m, 1H), 1.83-1.90 (m, 1H). LC-MS: 274 (MH)⁺.

Step E: Racemic (7,8-trans)-8-(4-fluorophenyl)-4-methoxy-5,6,7,8-tetrahydroquinolin-7-yl 3,5-5 bis(trifluoromethyl)benzoate

The title compound was prepared from racemic (7,8-trans)-8-(4-Fluorophenyl)-4-methoxy-5,6,7,8-tetrahydroquinolin-7-ol (step D) according to the procedure of Example 1, step F. ¹H-NMR (CDCl₃): δ: 8.40 (d, 1H, J=5.7 Hz), 8.38 (s, 2H), 8.07 (s, 1H), 7.08-7.11 (m, 2H), 7.01 (t, 2H, J=8.7 Hz), 6.74 (d, 1H, J=5.5 hz), 5.54-5.56 (m, 1H), 4.59 (d, 1H, J=4.3 Hz), 3.98 (s, 3H), 2.89-3.04 (m, 2H), 2.14-2.21 (m, 2H). LC-MS: 514 (MH)⁺.

Step F: Racemic (7,8-tians)-7-({1-[3,5-bis(trifluoromethyl)phenyl]vinyl}oxy)-8-(4-fluorophenyl)-4-methoxy-5,6,7,8-tetrahydroquinoline

The title compound was prepared from racemic (7,8-trans)-8-(4-fluorophenyl)-4-methoxy-5,6,7,8-tetrahydroquinolin-7-yl 3,5-bis(trifluoromethyl)benzoate (step E) according to the procedure of Example 1, step G. ¹H-NMR (CDCl₃): δ: 8.37 (d, 1H, J=5.7 Hz), 7.82 (s, 2H), 7.77 (s, 1H), 7.00-7.08 (m, 4H), 6.72 (d, 1H, J=5.7 Hz), 4.99 (d, 1H, J=3.4 Hz), 4.70-4.73 (m, 1H), 4.64 (d,, 1H, J=3.7 Hz), 4.61 (d, 1H, J=3.4 Hz), 3.96 (s, 3H), 2.82-2.92 (m, 2H), 2.10-2.20 (m, 2H). LC-MS: 512 (MH)⁺.

Step G: Racemic (7,8-trans)-7-{1-[3,5-bis(trifluoromethyl)phenyl]ethoxy}-8-(4-fluorophenyl)-4-methoxy-5,6,7,8-tetrahydroquinoline

The title compound was prepared from racemic (7,8-trans)-7-({1-[3,5-bis(trifluoro-methyl)phenyl]vinyl}oxy)-8-(4-fluorophenyl)-4-methoxy-5,6,7,8-tetrahydroquinoline (step F) according to the procedure of Example 1, step H. The crude title compounds were purified by prep TLC eluting with EtOAc/hexanes/2 N NH₃ in MeOH (10/10/0.5) to afford the two diastereomers. The less polar isomer, ¹H-NMR (CDCl₃):δ: 8.32 (d, 1H, J=5.5 Hz), 7.76 (s, 1H), 7.62 (s, 2H), 6.80-6.90 (m, 4 H), 6.66 (d, 1H, J=5.7 Hz), 4.74 (q, 1H, J=6.6 Hz), 4.26 (d, 1H, J=4.5 Hz), 3.94 (s, 3H), 3.70-3.73 (m, 1H), 2.75-2.93 (m, 2H), 1.92-2.07 (m, 2H), 1.46 (d, 3H, J=6.4 Hz). LC-MS: 514 (MH)⁺. The more polar isomer, ¹H-NMR (CDC13): δ: 8.37 (d, 1H, J=5.5 Hz), 7.81 (s, 1H), 7.78 (s, 2H), 6.96-7.00 (m, 4H), 6.68 (d, 1H,, J=5.7 Hz), 4.64 (q, 1H, J=6.4 Hz), 4.40 (d, 1H, J=4.8 Hz), 3.93 (s, 3H), 3.71-3.74 (m, 1H), 2.68-2.84 (m, 2H), 1.75-1.84 (m, 2H), 1.31 (d, 3H, J=6.4 Hz). LC-MS: 514 (MH)⁺.

Step H: Racemic (7,8-trans)-7-{1-[3,5-bis(trifluoromethyl)phenyl]ethoxy}-8-(4-fluorophenyl)-5,6,7,8-tetrahydroquinolin-4-ol or racemic (7,8-trans)-7-{1-[3,5-bis(trifluoromethyl)-phenyllethoxy}-8-(4-fluorophenyl)-5,6,7,8-tetrah vdroquinolin-4(1H)-one

A solution of 10 mg (0.019 mmol) of less polar isomer of racemic (7,8-trans)-7-{1-[3,5-bis(trifluoromethyl)phenyl]ethoxy}-8-(4-fluorophenyl)-4-methoxy-5,6,7,8-tetrahydroquinoline (less polar isomer of step G) in 3 mL DMF was added 13.6 mg (0.19 mmol) NaSMe. The reaction mixture was heated at 80° C. for 16 hr. The reaction mixture was cooled to RT and the solvent removed under vacuum. The residue was purified by reverse phase prep B :PLC (CH₃CN/water) to afford 8.3 mg of the title compound. ¹H-NMR (CDCl₃): δ: 7.85 (d, 1H, J=6.0Hz), 7.79 (s, 1H), 7.62 (s, 2H), 7.16 (d, 1H, J=6.0 Hz), 6.79-6.93 (m, 6H), 4.76 (q, 1H, J=6.6 Hz), 4.33 (d, 1H, J=4.1 Hz), 3.68-3.69 (m, 1H), 2.89 -2.94 (m, 2H), 2.01-2.05 (m, 2H), 1.46 (d, 3H, J=6.4 Hz). LC-MS: 500 (MH)⁺.

TABLE 1
The compounds in Table 1 were synthesized using
the foregoing methodology, but substituting the appropriately
substituted reagent as described in the foregoing examples.
The requisite starting materials were commercialy available,
described in the literature or readily synthesized by
one skilled in the art of organic
synthesis without undue experimentation.

| | | parent ion |
|---|---|---|
| Ex. # | Compound | (MH⁺) m/z |
| 9 | 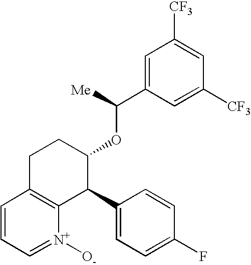 | 500 |
| 10 | 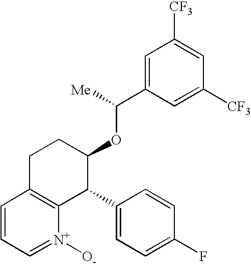 | 500 |
| 11 | 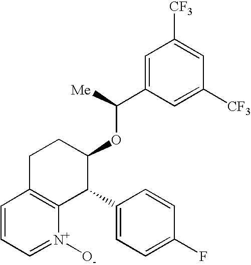 | 500 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound of the formula I: 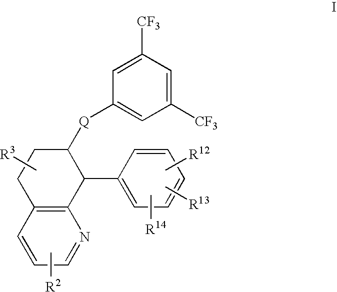
and N-oxides thereof,
wherein:
Q is selected from the group consisting of:
(1 ) —O—CH₂—,
(2) —O—CH(CH₃)—,
(3) —O—CH(CH₂OH)—,
(4) —O—(CO)—, and
(5) —O—(C═CH₂)—;
R² and R³ are independently selected from the group consisting of:
(1) hydrogen,
(2) C₁₋₆ alkyl, which is unsubstituted or substituted with one or more of the substituents selected from:
(a) hydroxy,
(b) oxo,
(c) C₁₋₆ alkoxy,
(d) phenyl-C₁₋₃ alkoxy,
(e) phenyl,
(f) halo,
(g) —NR⁹R¹⁰, wherein R⁹ and R¹⁰ are independently selected from:
(I) hydrogen,
(II) C₁₋₆ alkyl,
(III) phenyl,
(IV) (C₁₋₆ alkyl)-phenyl,
(V) (C₁₋₆ alkyl)-hydroxy, and
(VI) (C₁₋₆ alkyl)—(C₁₋₄ alkoxy), or where -NR⁹R¹⁰ forms a morpholine, piperidine or quinuclidine ring
(h) —NR⁹—COR¹¹, wherein R¹¹ is independently selected from:
(I) hydrogen,
(II) C₁₋₆ alkyl,
(III) phenyl,
(IV) (C₁₋₆ alkyl)-phenyl,
(V) (C₁₋₆ alkyl)-hydroxy, and
(VI) (C₁₋₆ alkyl)-(C₁₋₄ alkoxy),
(j) —NR⁹—CO₂R¹¹,
(k) —CO—NR⁹R₁₀,
(l) —COR¹¹,
(m) —CO₂R¹¹,
(3) hydroxy,
(4) C₁₋₆alkoxy,
(5) oxo,
(6) halo,
(7) —CN,
(8) —CF₃,
(9) —NR⁹R¹⁰,
(10) —NR⁹-COR¹¹,
(11) —NR⁹-CO₂R¹¹,
(12) —CO—NR⁹—COR¹¹,
(13) —COR¹¹,
(14) —O—(CO)R¹¹,
(15) —CO₂R¹¹,
(16) -imidazolyl, and
(17) -triazolyl;
R¹², R¹³ and R¹⁴ are independently selected from the group consisting of:
(1) hydrogen,
(2) halo, and
(3) C₁₋₆ alkyl;
and pharmaceutically acceptable salts thereof and individual enantiomers and diastereomers thereof.

2. The compound of claim 1 of the formula Ia: 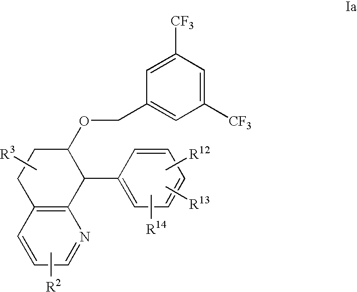
and N-oxides thereof, and pharmaceutically acceptable salts thereof and individual enantiomers and diastereomers thereof.

3. The compound of claim 2 of the formula Ia′: 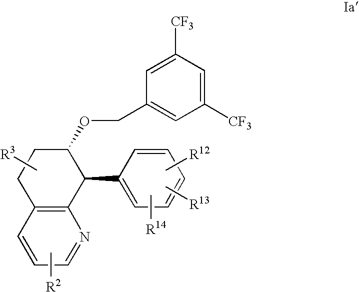
and N-oxides thereof, and pharmaceutically acceptable salts thereof and individual enantiomers and diastereomers thereof.

4. The compound of claim 1 of the formula Ib: 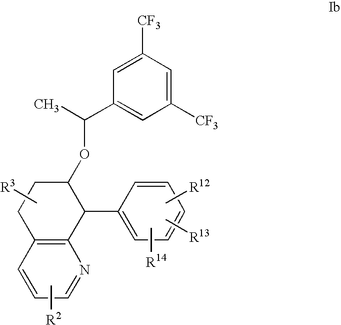
and N-oxides thereof, and pharmaceutically acceptable salts thereof and individual enantiomers and diastereomers thereof.

5. The compound of claim 4 of the formula Ib′: 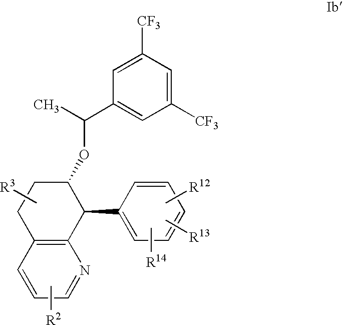
and N-oxides thereof, and pharmaceutically acceptable salts thereof and individual enantiomers and diastereomers thereof.

6. The compound of claim 1 wherein R² is selected from the group consisting of:
(1) hydrogen,
(2) C₁₋₆ alkyl, which is unsubstituted or substituted with one or more of the substituents selected from:
(a) morpholinyl,
(b) —NH₂,
(c) —NH(C₁₋₆ alkyl),
(d) —N(C₁₋₆ alkyl)(C₁₋₆ alkyl),
(e) hydroxy,
(f) —CO₂(C₁₋₆ alkyl),
(g) —N ECO(C₁₋₆ alkyl),
(h) —CO₂H, and
(i) triazolyl,
(3) hydroxy,
(4) halo,
(5) —CO₂(C₁₋₆ alkyl),
(6) —CO₂H, and
(7) —CN.

7. The compound of claim 6 wherein R² is hydrogen.

8. The compound of claim 6 wherein R² is methyl.

9. The compound of claim 1 wherein R³ is hydrogen.

10. The compound of claim 1 wherein R³ is fluoro.

11. The compound of claim 1 wherein R¹² is fluoro, R¹³ is hydrogen or methyl, and R¹⁴ is hydrogen.

12. The compound of claim 11 wherein R¹² is 4-fluoro, R¹³ is hydrogen and R¹⁴ is hydrogen.

13. The compound of claim 11 wherein R¹² is 4-fluoro, R¹³ is 2-methyl and R¹⁴ is hydrogen.

14. The compound of claim 1 wherein the compound is present as an N-oxide on the pyridyl ring.

15. A compound which is selected from the group consisting of:
(7,8-trans)-7-{ 1-[3,5-bis(Trifluoromethyl)phenyl]ethoxy}-8-(4-fluorophenyl)-5,6,7,8-tetrahydroquinoline;
(7R,8R)-7-{(1S)-1-[3,5-bis(Trifluoromethyl)phenyl]ethoxy}-8-(4-fluorophenyl)-5,6,7,8-tetrahydroquinoline;
(7S,8S)-7-{(1R)-1-[3,5-bis(trifluoromethyl)phenyl]ethoxy}-8-(4-fluorophenyl)-5,6,7,8-tetrahydroquinoline;
(7R,8R)-7-{(1R)-1-[3,5-bis(Trifluoromethyl)phenyl]ethoxy}-8-(4-fluorophenyl)-5,6,7,8-tetrahydroquinoline;
(7S,8S)-7-{(1S)-i -[3,5-bis(trifluoromethyl)phenyl]ethoxy}-8-(4-fluorophenyl)-5,6,7,8-tetrahydroquinoline;
(7S,8S)-7-{(1R)-1-[3,5-bis(trifluoromethyl)phenyl]ethoxy}-8-(4-fluorophenyl)-5,6,7,8-tetrahydroquinoline 1-oxide;
(7S,8S)-7-{(1 R)-1-[3,5-bis(Trifluoromethyl)phenyl]ethoxy} -2-chloro-8-(4-fluorophenyl)-5,6,7,8-tetrahydroquinoline;
(7S,8S)-7-{(1R)-1-[3,5-bis(Trifluoromethyl)phenyl]ethoxy}-8-(4-fluorophenyl)-5,6,7,8-tetrahydroquinolin-3-yl;
(7S,8S)-7-{(1R)-1-[3,5-bis(Trifluoromethyl)phenyl]ethoxy} -4-chloro-8-(4-fluorophenyl)-5,6,7,8-tetrahydroquinoline;
(7,8-trans)-7-{1-[3,5-bis(trifluoromethyl)phenyl]ethoxy}-8-(4-fluorophenyl)-5,6,7,8-tetrahydroquinolin-4-ol;
(7,8-trans)-7-{1-[3,5-bis(trifluoromethyl)phenyl]ethoxy}-8-(4-fluorophenyl)-5,6,7,8-tetrahydroquinolin-4(1H)-one; and pharmaceutically acceptable salts thereof.

16. A pharmaceutical composition which comprises an inert carrier and a compound of claim 1 or a pharmaceutically acceptable salt thereof.

17. (canceled)

18. (canceled)

19. A method for the treatment of pain or inflammation, migraine, emesis, postherpetic neuralgia, depression, anxiety or urinary incontinence, and LUTS which method comprises administration to a patient in need thereof a therapeutically effective amount of the compound of claim 1.

20. A method according to claim 16 for the treatment of urinary incontinence or LUTS.

21. A method of antagonizing the effect of substance P at its receptor site or for the blockade of neurokinin-1 receptors in a patient in need thereof comprising administration to said patient a therapeutically effective amount of the compound of claim 1.

22. A method of treating a physiological disorder associated with an excess of tachykinins in a patient in need thereof comprising administration to said patient a therapeutically effective amount of a compound of claim 1.

23. Use of a compound according to claim 1 for the manufacture of a medicament for antagonizing the effect of substance P at its receptor site or for the blockade of neurokinin-1 receptors in a mammal comprising combining a compound of the present invention or a pharmaceutically acceptable salt thereof with a pharmaceutical carrier or diluent.
